(19) United States
(12) Patent Application Publication
(10) Pub. No.: US 2010/0137581 A1
(43) Pub. Date: Jun. 3, 2010

(54) PROCESS FOR PREPARATION OF SUBSTANTIALLY PURE POLYMORPHIC FORM I OF OLANZAPINE

(75) Inventor: Tomasz Kozluk, Warszawa (PL)

Correspondence Address: MATTHIAS SCHOLL, 14781 MEMORIAL DRIVE, SUITE 1319, HOUSTON, TX, 77079, US

(73) Assignee: TOMASZ KOZLUK NOBILUS ENT, Warszawa (PL)

(21) Appl. No.: 12/523,983

(22) PCT Filed: Jan. 22, 2008
(86) PCT No.: PCT/PL08/00007

§ 371 Date: Jan. 21, 2010

(30) Foreign Application Priority Data
Jan. 22, 2007 (PL) P-381564

(51) Int. Cl. C07D 495/04

(52) U.S. Cl. 540/578

(57) ABSTRACT

A process for preparation of substantially pure polymorphic Form I of olazapine consists in that olanzapine starting material is subjected to: a) at least one digestion step in an acidic aqueous medium, with an optional addition of active charcoal, and then to neutralizing to pH within the range from about 6.0 to about 11.0, and b) at least one step of separation of impurities in a water—organic solvent system, followed by crystallization from the solution in methylene chloride, and wherein steps a) and b) are separated by steps of isolation of precipitate of olanzapine and can be accomplished in any order.

### FIELD OF INVENTION

The invention relates to a process for preparation of substantially pure polymorphic Form I of olanzapine. More particularly, the invention relates to the process for preparation of olanzapine comprising the step of purification of olanzapine followed by crystallization from the solution in methylene chloride.

### BACKGROUND OF THE INVENTION

2-methyl-4-(4-methyl-1-piperazinyl)-10H-thieno-[2,3b][1,5]benzodiazepine, is a known drug used in the treatment of schizophrenia and other disorders of central nervous system.

Olanzapine free base and acid addition salts thereof have been described in the European Patent Application EP-A-0454436.

One of the methods of preparation of olanzapine disclosed in EP-A-0454436 (Example 1.4) consists in a condensation of crude 4-amino-2-methyl-10H-thieno[2,3-b][1,5]benzodiazepine hydrochloride with N-methylpiperazine in a solution of dimethylsulfoxide and toluene, to which, upon completion of the reaction, water is added to isolate the product. The product prepared is crystallized from acetonitrile. In the second method (Example 2.3), olanzapine is prepared by effecting the ring closure of 1-{[2-(2-amino-anilino)-5-methylthiophen-3-yl]carbonyl}-4-methyl-piperazine, isolated by treating with ammonia, isopropanol and ethyl acetate, and purified by column chromatography on Florisil resin eluted with ethyl acetate and finally crystallized from acetonitrile. Both methods provide the product characterized by melting point 195° C.

European patent EP 0733635 B1 discloses a new polymorphic Form II of olanzapine and denominates the product obtained in EP-A-0454436 as Form I.

The term “polymorphic Form I of olanzapine” used hereinafter refers to 2-methyl-4-(4-methyl-1-piperazinyl)-10H-thieno-[2,3b][1,5]benzodiazepine as defined in EP-A-0454436 and characterized by the powder X-ray diffraction pattern obtained with a CuKα radiation source of wavelength λ=1,541×10⁻¹⁰ m, having the characteristic interplanar distances d, expressed in m⁻¹⁰: 9.9463, 8.5579, 8.2445, 6.8862, 6.3787, 6.2439, 5.5895, 5.3055, 4.9815, 4.8333, 4.7255, 4.6286, 4.533, 4.4624, 4.2915, 4.2346, 4.0855, 3.8254, 3.7489, 3.6983, 3.5817, 3.5064, 3.3392, 3.2806, 3.2138, 3.1118, 3.0507, 2.948, 2.8172, 2.7589, 2.6597, 2.6336 and 2.5956.

The term “crude olanzapine”, as defined in the International patent publication WO 96/38151, refers to 2-methyl-4-(4-methyl-1-piperazinyl)-10H-thieno-[2,3b][1,5]benzodiazepine associated with an undesired polymorphic form and/or containing more than about 5% of undesired related substances.

The term “technical olanzapine”, as defined in WO 96/38151, relates to 2-methyl-4-(4-methyl-1-piperazinyl)-10H-thieno-[2,3b][1,5]benzodiazepine, when no specific solvate or polymorphic form is named and containing less than 5% of undesired related substances.

In Preparation 1 of WO 96/38151, the polymorphic Form I of olanzapine is prepared by suspending technical olanzapine in methylene chloride, filtering the mixture and evaporating of a substantial part of the solvent from the resulting filtrate, filtering off and trituration of the wet precipitate with methylene chloride, and finally the two-step drying, on air and in vacuum oven at 50° C.

International patent publication WO 02/18390 discloses a process for preparation of the polymorphic Form I of olanzapine comprising crystallization of crude olanzapine, its hydrate or the polymorphic Form II from methylene chloride.

International patent publication WO 03/97650, also describes the method of preparation of the polymorphic Form I of olanzapine by crystallization of its solvates from methylene chloride. In one embodiment of the invention, olanzapine is extracted from a post-reaction mixture diluted with methylene chloride and water, then, a separated organic layer is concentrated and upon cooling to about 0-5□C, the resulting precipitate is isolated from the mixture, washed, macerated with methylene chloride, and then the wet precipitate of mixed solvate is crystallized from methylene chloride, to obtain the product containing less than 0.3% total impurities.

The processes disclosed in WO 02/18390 and WO 03/09765 applications comprise the preliminary purification of olanzapine preceding crystallization from a solution in methylene chloride with the use of active charcoal.

In the method described in WO 02/18390, raw olanzapine or the hydrate thereof or its polymorphic form II is dissolved in methylene chloride at reflux temperature, treated with active charcoal and filtered while hot, and not until that it is cooled and crystals are filtered off. The description does not provide any information on the purity level neither of the starting olanzapine nor of the product obtained.

International patent publication WO 2004/056833 reveals that in a process of crystallization of olanzapine from methylene chloride an additional impurity is formed, (1-(chloromethyl)-1-methyl-4-(2-methyl-10H-thieno[2,3-b][1,5]benzodiazepin-4-yl)piperazynium chloride, that is a product of quaternization of nitrogen atom of the piperazine ring with methylene chloride, referred to as “impurity S”. According to the authors of the application, that impurity cannot be efficiently removed by repeated crystallization or a standard treatment with active charcoal. On the contrary, extending duration of the process of crystallization leads to the accumulation of impurity S.

In view of the quality requirements concerning the pharmaceutically active substances determined by International Conference of Harmonization, there is a need of finding of the effective process for preparation of pharmaceutically pure olanzapine, in particular, olanzapine containing less than 0.15% impurity S.

That technical problem has been partially resolved by WO 2004/056833 application, wherein the solution of olanzapine in methylene chloride, prior to the crystallization, is treated with silica gel. Embodiments of the invention claimed in WO 2004/056833 comprise either passing the solution of olanzapine in methylene chloride through a column filled with the silica gel, which process is not suitable to be employed at industrial scale, or adding the silica gel to the solution. In the latter case, the treatment is performed at reflux temperature. Thus, the method involves laborious unit operations associated with treatment or filtration of the solution in methylene chloride while hot, which always takes a risk of entering the solvent vapor to the environment.

The method described in WO 2004/056833 allows to reduce impurity S content more than 10-fold, to a level of less than 0.15%, preferably less than 0.05%. However, in spite of the declared usefulness of the invention for purification even in case of the crude olanzapine, the claimed process makes it possible to increase the purity of olanzapine from the level 98.7% to 99.87% when passing through a column, and from 99% to 99.92% when adding silica gel to the solution.

Thus, none of the methods given in the prior art solves the technical problem of the purification of the crude olanzapine containing considerable amounts of impurities and related substances prior the crystallization from methylene chloride. In particular, none of the known methods teaches the pre-treatment of the crude olanzapine having the total amount of impurities and related substances going beyond 5%, especially from 5% to 50%, to be decreased to the level of each of them to 0.15% or less.

### DESCRIPTION OF FIGURES

FIG. 1A presents IR spectrum of polymorphic Form I of olanzapine prepared by the process of the invention; measurement range 400.0-4000.0 cm⁻¹.

FIG. 1B presents IR spectrum of polymorphic Form I of olanzapine prepared by the process of the invention; measurement range 400.0-1400.0 cm⁻¹.

FIG. 2 presents X-ray powder diffraction pattern of polymorphic Form I of olanzapine prepared by the process of the invention.

### DISCLOSURE OF THE INVENTION

The process for preparation of substantially pure polymorphic Form I of olanzapine consists in that olanzapine starting material is subjected to:
a) at least one step of digesting in an acidic aqueous medium, with optional adding active charcoal, and then neutralizing to pH from about 6.0 to about 11.0, and
b) at least one step of separation of impurities in a water—organic solvent system,
   followed by crystallization from the solution in methylene chloride, and wherein steps a) and b) are separated by steps of isolation of precipitate of olanzapine and can be accomplished in any order.

Whenever hereafter a reference is made to “substantially pure polymorphic Form I of olanzapine”, it is to be understood that it is a substance free of other polymorphic and/or pseudopolymorphic forms at amounts detectable with typical analytical methods, such as X-ray powder diffraction and solid state infrared absorption, i.e. containing less than 2%, generally less than 1% of other polymorphic and/or pseudopolymorphic forms, and moreover characterized by the content less than 0.15%, preferably less than 0.05% of each of the related substances, and in particular less than 0.15%, preferably less than 0.05% impurity S.

The starting material in the process of the present invention may be olanzapine prepared by any prior art method, e.g. the method described in EP 0454436 B1 or EP 0733635 B1, that involves reaction of condensation of molar excess of N-methylpiperazine with 4-amino-2-methyl-10H-thieno-[2,3-b][1,5]benzodiazepine hydrochloride in an organic solvent, e.g. dimethylsulfoxide or dimethylsulfoxide and toluene, and isolated from the post-reaction mixture upon diluting it with an excess of water.

The suitable acidic pH in the Step of digesting a) is ensured by a use of mineral acids, such as hydrochloric acid or sulfuric acid; organic acids, such as acetic acid; as well as acid anhydrides, such as maleic anhydride. Generally, a slight molar excess of an acid at concentration 0.1-5.0 wt % is used. Amount of the acid is adjusted according to the rules known to those skilled in the art, to ensure complete dissolution of olanzapine in an aqueous medium.

The neutralization of the reaction mixture in Step a) is accomplished with the use of a base selected from the carbonates and hydrogen carbonates of alkali metals and ammonium, as well as aliphatic amines. Examples of suitable amines are dimethylamine, diethylamine, diisopropylamine, triethanolamine and other.

Preferably, the neutralization is accomplished with the use of the carbonates and hydrogen carbonates of ammonium or sodium at amounts ensuring pH within the range about 6.0-9.0.

An organic solvent in Step b) is selected from the group comprising C₁-C₄-aliphatic ketones, such as acetone, methyl ethyl ketone, diisopropyl ketone; sulfoxides of lower alkyls C₁-C₄, such as dimethylsulfoxide; acyclic or cyclic tertiary amides of lower carboxylic acids C₁-C₃, such as N,N-dimethylformamide, N,N-diethylformamide, N,N-dimethylacetamide, N,N-diethylacetamide, N-methylpyrrolidone; aromatic carbohydrates, such as toluene, xylenes; aliphatic and C₁-C₇-cyclic ethers, such as tetrahydrofurane, dioxane; as well as esters of lower carboxylic acids and aliphatic alcohols C₁-C₄, such as ethyl acetate or butyl acetate; or their mixtures.

A number of steps necessary to carry out in a process of purification of olanzapine depends on purity of the starting material. The process according the present invention ensures advantageous results both when using technical olanzapine, crude olanzapine, as well as olanzapine recovered from the waste reaction liquors.

In one embodiment of the invention, crude olanzapine containing more than 5% impurities and related substances is used as the starting material.

In another embodiment of the invention, technical olanzapine containing less than 5% impurities and related substances is used as the starting material.

In still another embodiment of the invention, olanzapine recovered from the waste liquors of purity more than 50% is used as the starting material.

In the preferred embodiment, the first purification step carried out is digestion of olanzapine in an acidic aqueous medium (Step a). If a purity of olanzapine after the digestion step is higher than approx. 99.5%, one can pass directly to the step of separation of impurities in a water—organic solvent system. Otherwise, subjecting olanzapine to a subsequent step of digesting in an aqueous solution of an acid, and neutralizing, is preferable.

Upon the purification of olanzapine, the isolated precipitate is subjected to crystallization from methylene chloride. A single crystallization is usually sufficient to obtain the pharmaceutically pure olanzapine. Prior to crystallization, the olanzapine precipitate can be dried, although it is not necessary. If the wet precipitate of olanzapine is subjected to crystallization, a dehydrating agent is added to its solution in methylene chloride. Suitable dehydrating agents are, eg. molecular sieves, magnesium sulfate, sodium sulfate or calcium chloride.

In case of carrying out two subsequent steps of digestion and one step of separation of impurities in water—organic solvent system, upon final crystallization from methylene chloride, olanzapine of purity (HPLC) more than 99.85% is prepared.

The resulting product is analyzed by Fourier Transform Infrared spectroscopy (FTIR), X-ray powder diffraction (XRPD) and high-performance liquid chromatography (HPLC) methods.

FTIR spectra of polymorphic forms of olanzapine recorded using KBr pellets technique are, according to WO 03/097650, a suitable method to allow distinguishing polymorphic forms of olanzapine and determination of its content in the mixtures. FTIR spectra of polymorphic Forms I and II of olanzapine distinctly differ between each other, in particular within the range of N-H vibrations. The greatest differences in spectra are exhibited in the following ranges: 3320-3000, 1680-1490, 980-950, 920-860, 780-730 cm⁻¹, and particularly within the range 920-860 cm⁻¹. It is associated with occurrence of hydrogen bonding of different strength in crystals of various polymorphic forms of olanzapine.

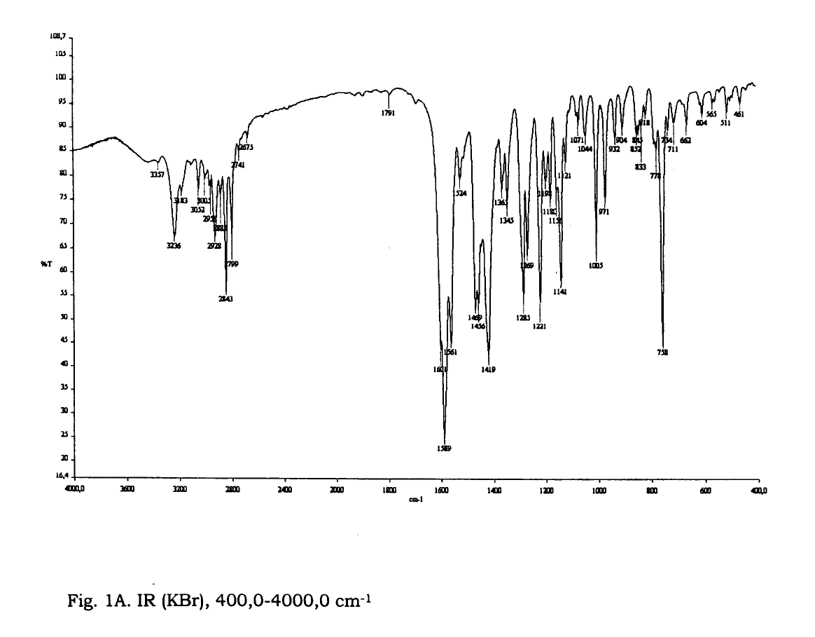

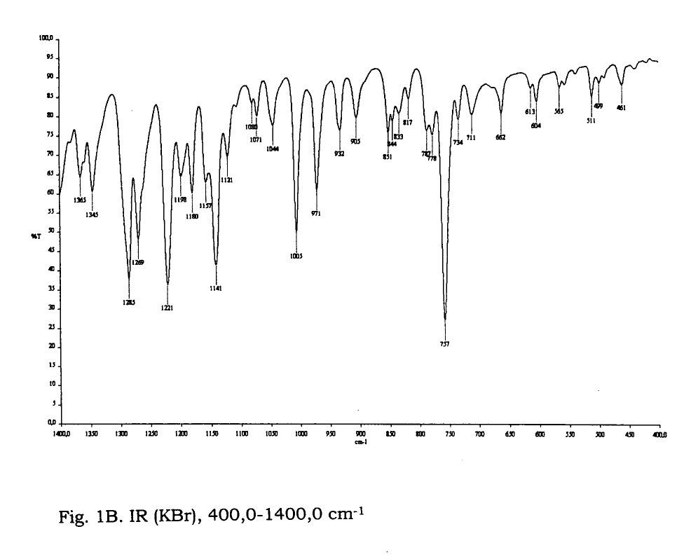

Exemplary FTIR spectrum of polymorphic form of olanzapine prepared by the process of the invention, recorded using Perkin Elmer Spectrum BX spectrometer is presented in FIGS. 1A and 1B.

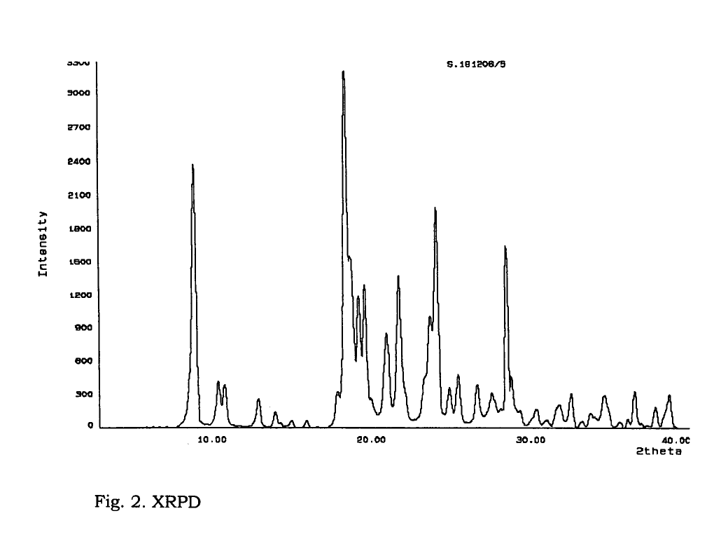

Exemplary X-ray powder diffractogram of polymorphic form of olanzapine prepared by the process of the invention, recorded with a Rigaku MINI FLEX diffractometer in the range of 3-40° in 2θ (deg, for CuKα, λ=1,542 Å; scanning rate 0.5 deg/min, scanning step 0.03 deg) is presented as the interplanar spacings d versus the relative intensity I/I₀ (expressed as a percentage of the most intense reflection) in FIG. 2.

The present invention provides an efficient method of preparation of polymorphic form I of olanzapine that makes it possible to reduce a content of impurities to a level complying with ICH requirements. In particular, the invention enables obtaining olanzapine containing less than 0.15% impurity S, preferably less than 0.05% impurity S. The method is straightforward for implementation at industrial scale at ambient temperature and eliminates necessity of using laborious operations associated with processing and filtrating methylene chloride solutions while hot. It makes possible to purify the product starting from olanzapine of any degree of purity.

The invention will now be illustrated by the following examples, which should not be construed as any limitation of its scope.

### Example 1

### Step I

To a flask containing 4 L of 0.1 wt % hydrochloric acid, 300 g (0.96 mole) of technical olanzapine (96.44%, HPLC) is added and the resulting mixture is stirred at room temperature until complete digestion of olanzapine takes place (30 min). Then, 30 g of active charcoal is added, the reaction mixture is stirred for further 10 min and then filtered. To a filtrate, 150 g (1.9 mole) of ammonium hydrogen carbonate, is portionwise added until pH approx. 9.0. The resulting mixture is stirred for 60 min, and then a precipitate is collected and washed with water. The resulting product is dried in vacuo at 70° C. for 12 hours.

Olanzapine (270 g) of purity 99.16% (HPLC) is obtained.

### Step II

The product resulting from Step I is digested in 4 L of 0.1 wt % hydrochloric acid, 27 g of active charcoal is added, the resulting mixture is stirred for 10 min and then filtered. To a filtrate, 150 g of ammonium hydrogen carbonate is portionwise added to pH 8. The resulting precipitate is filtered off, washed with water and dried at 70° C. in vacuo for 12 h. Olanzapine (249 g) of purity 99.67% (HPLC) is obtained.

### Step III

Olanzapine (10.9 g) obtained in Step II is dissolved in DMSO (22 g), then water (40 mL) is dropwise added while stirring, and additionally 50 mL of water is poured. The resulting precipitate is filtered off, washed with water and dried at 70° C. for 12 h. Olanzapine (10.7 g) of purity 99.89% (HPLC) is obtained. Impurity S content: 0.047%.

### Step IV

A. The compound prepared in Step III (3 g) dissolved in methylene chloride (60 mL) with an addition of calcium chloride (0.6 g), is stirred for 15 min. The solution is filtered. The filtrate is concentrated in vacuo at 25° C. until volume of 12 mL and allowed to crystallize at 4° C. The resulting precipitate is collected, washed with chilled methylene chloride, and dried in vacuo at 70° C. for 12 h. The product identified by X-ray powder diffraction and IR spectroscopy as olanzapine polymorphic Form I is obtained with the yield 2.52 g. Purity: 99.91% (HPLC). Impurity S content: 0.044%.

M.p. 193-194° C. (uncorr.).

B. Compound obtained in Step III is subjected to an additional drying step in vacuo at 70° C. for 12 h. Then, 3 g of the product is taken and dissolved in hot methylene chloride at 60° C. The solution is concentrated in vacuo at 25° C. until volume of 12 mL and left for 12 h at 4° C. The precipitate is filtered off. The product identified by X-ray powder diffraction and IR spectroscopy as olanzapine polymorphic Form I is obtained with the yield 1.77 g. Purity: 99.91% (HPLC). Impurity S content: 0.044%.

M.p. 193-194° C. (uncorr.).

Examples 2 to 4 illustrate a use of various acids in the step a), Examples 5 to 8 illustrate a use of various organic solvents in the step b), and Example 9 pertains to the process of purification of olanzapine of purity of the starting material approx. 55% (HPLC).

### Example 2

### Step I

Olanzapine (20.0 g, 0.064 mole) of purity 95.058% (HPLC) is transferred to a flask containing a solution of acetic acid, the resulting mixture is stirred at room temperature until complete digesting of olanzapine. Then, active charcoal is added, the resulting mixture is stirred and then filtered. To a filtrate, ammonium hydrogen carbonate is portionwise added while continuing stirring. The resulting precipitate is filtered off and washed with water. The obtained product is dried in vacuo at 70° C. for 24 h. Olanzapine of purity 98.55% (HPLC) is obtained.

### Step II

The product obtained in the previous step (10 g) is digested in 1.5 wt % acetic acid (400 mL), active charcoal is added and the resulting mixture is stirred and then filtered. To a filtrate, ammonium hydrogen carbonate is portionwise added to pH 9. The resulting precipitate is filtered off and washed with water. Then, it is dried in vacuo at 70° C. for 24 h. Olanzapine (9.81 g) of purity 99.27% (HPLC) is obtained.

### Example 3

### Step I

Olanzapine (20.0 g, 0.064 mole) of purity 95.058% (HPLC) is transferred to a flask containing 400 mL of diluted sulfuric acid, and the resulting mixture is stirred at room temperature until complete digesting of olanzapine (30 min.). Then, 2 g of active charcoal is added, the resulting mixture is stirred for further 10 min., and then the mixture is filtered. To a filtrate, ammonium hydrogen carbonate is portionwise added and the mixture stirred. The resulting precipitate is filtered off and washed with water. The resulting compound is dried in vacuo at 70° C. for 12 h. Olanzapine (18.3 g) of purity 97.95% (HPLC) is obtained.

### Step II

The product obtained in the previous step (14 g) is digested in 400 mL of dilute sulfuric acid, 2 g of active charcoal is added, the mixture is stirred, and then filtered. To a filtrate, ammonium hydrogen carbonate is portionwise added to pH 7. The resulting precipitate is filtered off, washed with water, and dried in vacuo at 70° C. for 12 h. Olanzapine (10.8 g) of purity 99.25% (HPLC) is obtained.

### Example 4

### Step I

Olanzapine (20.0 g) of purity 94.60% (HPLC) is placed in a flask containing 400 mL of water, maleic anhydride (6 g) is added and the resulting mixture is stirred at room temperature until complete digesting of olanzapine (30 min.). Then, active charcoal is added, the resulting mixture is stirred for further 10 min., and then it is filtered. To a filtrate, ammonium hydrogen carbonate is portionwise added to pH 8 and the mixture is stirred for 60 min. The resulting precipitate is filtered off and washed with water. The solid is dried in vacuo at 70° C. for 24 h. Olanzapine of purity 98.16% (HPLC) is obtained.

### Step II

Product obtained in Step I (10 g) is digested in 400 mL of water, then maleic anhydride and active charcoal are added, the reaction mixture is stirred for 10 min, then filtered off and to a filtrate ammonium hydrogen carbonate is portionwise added (pH 8). The resulting precipitate is filtered off, washed with water and dried in vacuo at 70° C. for 24 h. Olanzapine (10.98 g) of purity 99.53% (HPLC) is obtained.

### Example 5

Olanzapine (1 g) of purity 98.85% (HPLC) is dissolved in DMSO (7 mL), then, water is added while stirring. The resulting precipitate is filtered off, washed with water and dried at 70° C. for 24 h. Olanzapine (0.88 g) of purity 99.43% (HPLC) is obtained.

### Example 6

Olanzapine (1 g) of purity 98.85% (HPLC) is dissolved in DMF (8 mL), and then water is added while stirring. The resulting precipitate is filtered off, washed with water and dried for 24 h at 70° C. Olanzapine (0.82 g) of purity 99.58% (HPLC) is obtained.

### Example 7

Olanzapine (1 g) of purity 98.85% (HPLC) is dissolved in 15 mL of acetone, then water is added while stirring. The resulting precipitate is filtered off, washed with water and dried for 24 h at 70° C. Olanzapine (0.82 g) of purity 99.65% (HPLC) is obtained.

### Example 8

Olanzapine (1 g) of purity 98.85% (HPLC) is dissolved in ethyl acetate (15 mL), then water is added while stirring. The resulting precipitate is filtered off, washed with water and dried for 24 h at 70° C. Olanzapine (0.62 g) of purity 99.63% (HPLC) is obtained.

### Example 9

### Step I

Olanzapine (100.0 g, 0.32 mole) of purity 55.04% (HPLC) is placed in a flask containing 1.3 L of 0.1% hydrochloric acid and the resulting mixture is stirred at room temperature until complete digesting of olanzapine (30 min). Then, 10 g of active charcoal is added, the mixture is stirred for further 10 min, and then it is filtered. To a filtrate, 50 g of ammonium hydrogen carbonate is portionwise added (pH 8). Then, the resulting mixture is stirred for 60 min. After that time, the resulting precipitate is filtered off, washed with water and dried in vacuo at 70° C. for 12 h. Olanzapine (43.1 g) of purity 93.16% (HPLC) is obtained.

### Step II

The compound obtained in Step I is digested in 0.6 L of 0.1% hydrochloric acid and 4 g of active charcoal is added, the resulting mixture is stirred for 10 min and then filtered. To a filtrate, 30 g of ammonium hydrogen carbonate is portionwise added (pH 9). The resulting precipitate is separated, washed with water and dried in vacuo in 70° C. for 12 h. Olanzapine (37.9 g) of purity 98.91% (HPLC) is obtained.

### Step III

The compound obtained in Step II is digested in 0.5 L of 0.1% hydrochloric acid and 3.5 g of active charcoal is added, the resulting mixture is stirred for 10 min, and then filtered. To a filtrate, 29 g of ammonium hydrogen carbonate is portionwise added (pH 8). The resulting precipitate is separated, washed with water and dried in 70° C. in vacuo for 12 h. Olanzapine (35.3 g) of purity 99.61% (HPLC) is obtained.

### Step IV

From olanzapine obtained in Step III, a sample 10.9 g is taken and dissolved in 22 g DMSO; then, 40 mL of water is dropwise added while stirring and additional 50 mL is poured. The resulting precipitate is filtered off and washed with water. Then, it is dried at 70° C. for 48 h. 10.7 g of olanzapine of purity 99.89% (HPLC) is obtained. Impurity S content: 0.048%.

What is claimed is:

1. A process for preparation of substantially pure polymorphic Form I of olanzapine, characterized in that olanzapine starting material is subjected to:
a) at least one digestion step in an acidic aqueous medium, with optional addition of active charcoal, and then neutralizing to pH within the range from about 6.0 to about 11.0, and
b) at least one step of impurities separation in a water—organic solvent system,
followed by crystallization from the solution in methylene chloride,
and wherein the steps a) and b) are separated by steps of isolation of precipitate of olanzapine and can be accomplished in any order.

2. The process according to claim 1, characterized in that olanzapine is digested in an aqueous solution of a mineral acid, an organic acid or an acid anhydride.

3. The process according to claim 1, characterized in that a molar excess of an acid in relation to olanzapine is used.

4. The process according to claim 2, characterized in that olanzapine is digested in an aqueous solution of hydrochloric acid.

5. The process according to claim 1, characterized in that neutralization is accomplished with the use of an inorganic or organic base.

6. The process according to claim 1, characterized in that neutralization is accomplished with the use of the inorganic base selected from carbonates and hydrogen carbonates of alkali metals and ammonium, or aliphatic amines.

7. The process according to claim 6, characterized in that neutralization is accomplished with the use of ammonium or sodium carbonate or hydrogen carbonate.

8. The process according to claim 1, characterized in that pH is neutralized to the range 6.0-9.0.

9. The process according to claim 1, characterized in that the organic solvent is selected from the group comprising C₁-C₄-aliphatic ketones, such as acetone, methyl ethyl ketone, diisopropyl ketone; sulfoxides of lower alkyls C₁-C₄, such as dimethylsulfoxide; acyclic or cyclic tertiary amides of lower carboxylic acids C₁-C₃, such as N,N-dimethylformamide, N,N-diethylformamide, N,N-dimethylacetamide, N,N-diethylacetamide, N-methylpyrrolidone; aromatic carbohydrates, such as toluene, xylenes; C₁-C₄-aliphatic and C₁-C₇-cyclic ethers, such as tetrahydrofuran, dioxane; as well as esters of lower carboxylic acids and aliphatic alcohols C₁-C₄, such as ethyl acetate or butyl acetate; or their mixtures.

10. The process according to claim 1, characterized in that the starting material is crude olanzapine containing more than 5% related substances.

11. The process according to claim 1, characterized in that the starting material is the technical olanzapine containing less than 5% related substances.

12. The process according to claim 1, characterized in that the starting material is olanzapine recovered from the waste liquor of purity more than 50%.

13. The process according to claim 1, characterized in that upon initial purification, the precipitate of olanzapine is subjected to a single-step crystallization from methylene chloride.

14. The process according to claim 13, characterized in that the wet precipitate of olanzapine is subjected to crystallization, wherein the crystallization is carried out with the addition of a dehydrating agent.

15. The process according to claim 14, characterized in that the dehydrating agent is selected from the group comprising molecular sieves, magnesium sulfate, sodium sulfate and calcium chloride.

16. The process according to claim 13, characterized in that the dried precipitate of olanzapine is subjected to crystallization.

17. Substantially pure polymorphic Form I of olanzapine containing less than 0.10% impurity S, prepared by the method according to any of the preceding claims.

18. Substantially pure polymorphic Form I of olanzapine containing less than 0.05% impurity S, prepared by the method according to any of the claims 1-16.
